(19) United States
(12) Patent Application Publication
(10) Pub. No.: US 2022/0249052 A1
(43) Pub. Date: Aug. 11, 2022

(54) Method for calculating density images in a human body, and devices using the method

(71) Applicant: Akihiro Haga, Tokushima (JP)

(72) Inventors: Akihiro Haga, Tokushima (JP); Taisei Shimomura, Tokushima (JP)

(73) Assignee: Akihiro Haga, Tokushima (JP)

(21) Appl. No.: 17/584,380

(22) Filed: Jan. 26, 2022

(30) Foreign Application Priority Data
Feb. 9, 2021 (JP) 2021-71432
Nov. 22, 2021 (JP) 2021-202061
Dec. 14, 2021 (JP) 2021-215519

(51) Int. Cl. A61B 6/00; A61B 6/03; G06T 7/44

(52) U.S. Cl. CPC A61B 6/583; A61B 6/4085; A61B 6/4233; A61B 6/482; G06T 2207/10081; A61B 6/032; G06T 7/44; G06T 2207/20081; G06T 2207/20084; A61B 6/4035

(57) ABSTRACT

Density images of electrons and/or elements for a large number of different virtual human phantoms were generated. Subsequently, a large number of x-ray projection images of said virtual human phantoms were calculated. Next, deep learning for a multi-layered neural network was performed using said x-ray projection images as input training data and said density images as output training data. Finally, density images of a new human body were obtained by inputting x-ray projection images of said new human body to the trained multi-layered neural network (FIG. 4).

### TECHNICAL FIELD

The present invention relates to a method for estimating density distributions of electrons and/or elements in a human body using X-ray computer tomography (CT) unit in general and more particularly X-ray cone-beam CT unit.

### BACKGROUND ART

It was difficult to accurately localize a tumor inside a human body using a mark drawn on a body surface. It was known that a linac with an X-ray cone-beam CT unit could improve the accuracy in tumor localization, which was disclosed in U.S. Pat. No. 6,842,502B2 entitled “Cone beam computed tomography with a flat panel imager”, the disclosure of which is hereby incorporated by reference.

X-ray beams generated by an X-ray source in the X-ray cone-beam CT unit pass through a patient body and reach a two-dimensional flat panel detector thereby producing projection images. The detector also receives scattered X-rays produced inside the patient body. The scattered X-ray signals were not needed to reconstruct cone-beam CT images, which was also known to degrade the image contrast of the projection images as well as reconstructed cone-beam CT images.

The method for reconstructing a cone-beam CT volumetric (3D) image from projection images (2D) was disclosed in Feldkamp L A, Davis L C and Kress J W 1984 Practical cone-beam algorithm. J. Opt. Soc. Am. A 1 612-9, haps://doi.org/10.1364/JOSAA.1.000612, the disclosure of which is hereby incorporated by reference. This method was known as Feldkamp's back projection, where cone-beam projections from many beam angles were backward-projected in order to create a three-dimensional (3D) volume in a patient. This algorithm has been widely used in various industries.

Contrast degradation in the cone-beam CT images caused by the scattered X-rays was known to make the soft tissue contouring difficult. A grid for reducing the scattering was also reported; however, the grid alone would not sufficiently reduce the scattering and therefore a more effective method has been awaited.

A deep-learning based method was also disclosed in Kida S, Nakamoto T, Nakano M, et al. Cone Beam Computed Tomography Image Quality Improvement Using a Deep Convolutional Neural Network. Cureus 10: e2548, 2018. doi: 10.7759/cureus.2548, the disclosure of which is hereby incorporated by reference. In this method, a paired set of treatment planning CT images having much less scattered X-ray components and cone-beam CT images were collected from many patients. Then, these images were fed into a multi-layered neural network for deep learning or training. After the learning or training was completed, a new cone-beam CT image was inputted to the trained neural network, resulting in a scatter-free CT image as an output. In other words, the neural network was configured to remove scattering components from the cone-beam CT image and thus provided improved contrast similar to treatment planning CT images. The problem may be that a large number of paired sets of patient images need to be collected, which may require a lot of time. The above reference also reported that resulting outputs from the trained neural network might not be reliable possibly due to misplacement between the corresponding treatment planning CT and cone-beam CT images.

### SUMMARY

The present invention employs a large number of different virtual human phantoms that are generated in a computer. Subsequently, a paired set of X-ray projection images and density images are calculated by referring to each of the generated virtual human phantoms. Then, deep learning of a multi-layered neural network is performed based on the projection images as input training data and corresponding density images as output training data, both from the identical virtual human phantom. After training is completed, a density image is estimated by inputting projection images of a new patient to the trained multi-layered neural network. This approach can solve two major problems described in the background art: i) The misplacement between paired image data does not happen because only virtual human phantoms are used to generate the paired image data, ii) Time-consuming paired image data collection is not required from a large number of patients because virtual human phantoms are generated in a computer.

In accordance with one embodiment, density images of electrons and/or elements for a large number of virtual human phantoms having varied shape and material properties are generated. Subsequently, a large number of X-ray projection images of said virtual human phantoms are calculated. Next, deep learning for a multi-layered neural network is performed using said X-ray projection images as input training data and said density images as output training data. Finally, density images of a new human body are obtained by inputting X-ray projection images of said new human body to the trained multi-layered neural network.

In accordance with another embodiment, density images of electrons and/or elements for a large number of different virtual human phantoms are generated. Subsequently, a large number of X-ray projection images of said virtual human phantoms are calculated. Then, cone-beam CT images are reconstructed based on the X-ray projection images for each of the large number of virtual human phantoms. Next, deep learning for a multi-layered neural network is performed using said cone-beam CT images as input training data and said density images as output training data. Finally, density images of a new human body are obtained by inputting cone-beam CT images of said new human body to the trained multi-layered neural network

### Advantages

In the present invention, a large number of virtual human phantoms with known density and/or material distributions are generated in a computer, which are used for deep learning of a multi-layered neural network. This approach can disregard the misplacement issue between paired images because an identical virtual human phantom is used for specifying both input and output training data. In addition, in the virtual human phantoms, various phantom parameters are statistically varied thereby producing a large number of different vitual human phantoms. In other words, a large number of paired images (such as density image and cone-beam CT image) are efficiently generated, thereby accelerating training process. This implies that conversion from a new cone-beam CT image to scatter-free CT image is more accurately performed. For example, contours of a tumor and critical organs on the day are efficiently and accurately extracted while a patient is placed on a treatment couch; subsequently, a treatment plan can be optimized immediately before every treatment fraction starts. Even when the tumor and nearby critical organs are significantly deformed or displaced, online adaptive treatment can provide highest possible local tumor control without increasing toxicity to critical organs. In addition, material parameters such as shape, density, and element composition can be largely varied according to given statistics such as Gaussian distributions. In the deep learning theory, an estimated output by a neural network may become unstable when the input was outside the trained parameter space. In other words, the input data space for the training needs to be large enough to obtain a reliable output. Another advantage of this invention is that dose distributions can be more accurately calculated because accurate density distributions are obtained.

### DESCRIPTION OF DRAWINGS

FIG. 1 illustrates an example radiotherapy system equipped with a cone-beam CT unit (9, 11), according to some embodiments of the present disclosure.

FIG. 2 shows X-ray transport after emitted from an X-ray source 9, according to some embodiments of the present disclosure.

FIG. 3 illustrates an example diagnostic X-ray CT unit, according to some embodiments of the present disclosure.

FIG. 4 shows a flowchart that describes a method for calculating an scatter-free X-ray cone-beam CT image or a density image, according to some embodiments of the present disclosure.

FIG. 5 is a diagram that shows a method for calculating a direct X-ray projection image of a virtual human phantom 22, according to some embodiments of the present disclosure.

FIG. 6 demonstrates a set of element density images in a human body, comprising hydrogen (H), carbon (C), nitrogen (N), oxygen (O), phosphorus (P), and calcium (Ca), according to some embodiments of the present disclosure.

FIG. 7 is a trajectory of scattered X-ray beams 27 reaching a flat panel detector 11, according to some embodiments of the present disclosure.

FIG. 8 shows a flowchart for calculating X-ray projection images, according to some embodiments of the present disclosure.

FIG. 9 is an example X-ray spectrum obtained in the step 1 of FIG. 8, according to some embodiments of the present disclosure.

FIG. 10 is an example projection image of a virtual human phantom calculated by the flowchart in FIG. 8, according to some embodiments of the present disclosure.

FIG. 11 is a block diagram for performing step 1 through STEP 3 of FIG. 4, according to some embodiments of the present disclosure.

FIG. 12 is a block diagram for performing STEP 4 of FIG. 4, according to some embodiments of the present disclosure.

FIG. 13 is another flowchart that describes a method for obtaining density images which is regarded as scatter free X-ray cone-beam CT images, according to some other embodiments of the present disclosure.

FIG. 14 is an example cone-beam CT image reconstructed by STEP 3 of FIG. 13, viewed as three orthogonal slice images, according to some embodiments of the present disclosure.

FIG. 15 is a block diagram that shows a deep learning process of a multi-layered neural network 49 with input training data of cone-beam CT images 47 and output training data of density images 43, according to some embodiments of the present disclosure.

FIG. 16 is a block diagram showing that a cone-beam CT image 61 of a new human body is fed into the trained neural network 49A, leading to density images 65 of electrons and/or elements of the same human body, according to some embodiments of the present disclosure.

FIG. 17 is example density images of the elements in the human body as an output from the trained multi-layered neural network 49A shown in FIG. 16, according to some embodiments of the present disclosure.

FIG. 18 is a flowchart showing a new method for obtaining an X-ray spectrum of an X-ray source, according to some embodiments of the present disclosure.

FIG. 19 is a block diagram that shows a deep learning process from STEP 1 through STEP 4 of FIG. 18, according to some embodiments of the present disclosure.

FIG. 20 is a block diagram that provides an X-ray spectrum 75 as an output of a trained neural network 69A as shown in the STEP 5 of FIG. 18 after inputting a cone-beam CT image 71 of a new human body, according to some embodiments of the present disclosure.

FIG. 21 is a diagram showing a method for calculating a direct X-ray projection image of a virtual human phantom 22 with a bowtie filter 10 placed near the X-ray source 9, according to some embodiments of the present disclosure.

FIG. 22 is a perspective view of a typical bowtie filter 10, according to some embodiments of the present disclosure.

FIG. 23 is a flowchart that shows a method for calculating an X-ray spectrum when a bowtie filter is placed with known shape and material information, according to some embodiments of the present disclosure.

FIG. 24 is a block diagram of a deep learning process shown as STEP 1 through STEP 4 of FIG. 23, according to some embodiments of the present disclosure.

FIG. 25 is a block diagram for obtaining an X-ray spectrum 75 by inputting a cone-beam CT image 71A of a new human body to a trained neural network 73A as shown in STEP 5 of FIG. 23, according to some embodiments of the present disclosure.

FIG. 26 is a flowchart that shows a method for calculating an X-ray spectrum when a bowtie filter with unknown shape and material information is employed, according to some embodiments of the present disclosure.

FIG. 27 is a block diagram of a deep learning process shown as STEP 1 through STEP 4 of FIG. 26, according to some embodiments of the present disclosure.

FIG. 28 is a block diagram for obtaining a cone-angle dependent X-ray spectrum 82 of the beam 23A after passing the bowtie filter 10, by inputting a cone-beam CT image 80 of a new human body to the trained multi-layered neural network 77A as shown in STEP 5 of FIG. 26, according to some embodiments of the present disclosure.

FIG. 29 is a flowchart showing a method for calculating a density image with an organ label image by calculating cone-beam CT images of a large number of virtual human phantoms, according to some embodiments of the present disclosure.

FIG. 30 is a block diagram of a deep learning process shown as STEP 1 through STEP 4 of FIG. 29, according to some embodiments of the present disclosure.

FIG. 31 is a block diagram showing the calculation process for the STEP 5 of FIG. 29, according to some embodiments of the present disclosure.

FIG. 32 is a flowchart for calculating a set of density images and organ label images based on projection images of a large number of virtual human phantoms, according to some embodiments of the present disclosure.

FIG. 33 is a block diagram of a deep learning process as shown in STEP 1 through STEP 3 of FIG. 32, according to some embodiments of the present disclosure.

FIG. 34 is a block diagram showing a calculation process of STEP 4 of FIG. 32, according to some embodiments of the present disclosure.

FIG. 35 is an example cone-beam CT image 47 in FIG. 30, which is calculated by the procedure shown in FIG. 29, according to some embodiments of the present disclosure.

FIG. 36 is example element density images 88B with a corresponding organ label image 88A, both of which are provided for deep learning shown in FIG. 30 or FIG. 33, according to some embodiments of the present disclosure.

### REFERENCE NUMERALS IN THE DRAWINGS

1 gantry head
3 collimator
5 gantry rotating means
7 patient couch
9 X-ray source
10 bowtie filter
11 flat panel detector
12 flat panel detector for treatment beams
13 display unit
15 control computer
17 control signal cable
19 human body
21 virtual human phantom
23 direct X-ray beam
23A direct X-ray beam after passing a bowtie filter
25 X-ray beam before scattering
27 scattered X-ray beam
29 detector
31 fan beam X-ray
33 detector element in a flat panel detector
35 voxel j
41 X-ray projection images
41A X-ray projection image with a bowtie filter placed
43 density images of electrons and/or elements
45 multi-layered neural network for deep learning
45A trained multi-layered neural network after deep learning is completed
47 X-ray cone-beam CT images
47A cone-beam CT images with a bowtie filter placed
49 multi-layered neural network for deep learning
49A trained multi-layered neural network after deep learning is completed
51 X-ray projection images of a new human body
55 density images of electrons and/or elements
61 X-ray cone-beam CT image of a new human body
65 density images of electrons and/or elements
67 randomly sampled X-ray spectrums
69 multi-layered neural network for deep learning
69A trained multi-layered neural network after deep learning is completed
71 X-ray cone-beam CT image of a new human body
71A X-ray cone-beam CT image of a new human body with a bowtie filter placed
73 multi-layered neural network for deep learning with a bowtie filter
73A trained multi-layered neural network after deep learning with a bowtie
filter is completed
75 estimated X-ray spectrum
77 multi-layered neural network for deep learning
77A trained multi-layered neural network after deep learning is completed
78 a large number of bowtie filter models
82 cone-angle dependent X-ray spectrums of beam 23A after passing the bowtie
filter
84 multi-layered neural network for deep learning
84A trained multi-layered neural network after deep learning is completed
85 multi-layered neural network for deep learning
85A trained multi-layered neural network after deep learning is completed
86 density images of elements with an organ label image corresponding to a cone-beam CT image 51 of a new human body
88 density images of elements with organ label images
88A organ label images
88B element density images (density images of each of the elements in a human body) that is linked to an organ label image

Suitable embodiments of a method for calculating density images in a human body, and devices using the method according to the present invention will be described in the following details with reference to the attached drawings.

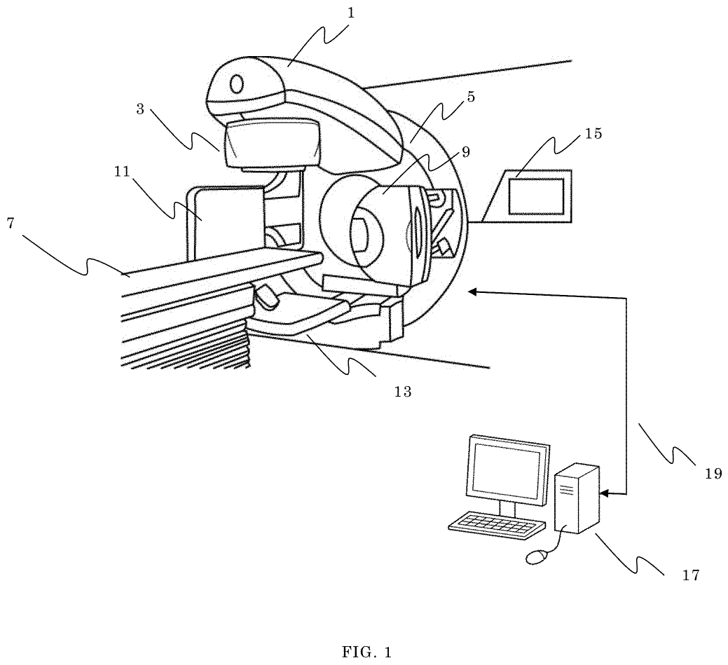

### Detailed Description: First Embodiment with FIGS. 1-12

FIG. 1 illustrates an example radiotherapy system according to the first embodiment of the present disclosure, having a gantry head 1 that generates treatment X-ray beams, a collimator unit 3 that shapes the treatment X-ray beam to fit a tumor shape, a gantry rotating means 5 that specifies the direction of the treatment beams delivered from the gantry head, a patient couch 7 that places a tumor at the treatment X-ray beam position, an X-ray source 9 and a flat panel detector 11 to produce a cone-beam CT image, another flat panel detector 13 for treatment X-ray beams, and a display unit 15 that shows system operating status. The X-ray source 9 includes an X-ray tube, a filter, and a collimator. A control computer 17 is placed at an operation room adjacent to a treatment room where a radiotherapy system is installed. A control signal cable 19 connects the control computer 17 to the radiotherapy system. The computer 17 controls the entire radiotherapy system and also contains a cone-beam CT reconstruction program. An X-ray projection image measured by the flat panel detector 11 is sent to the computer 17 via the control signal cable 19. By activating the gantry rotating means 5, a cone-beam CT image is reconstructed by acquiring a number of X-ray projection images with different gantry angles, which are measured by the flat panel detector 11 during gantry rotation. In the present patent application, X-ray cone-beam CT unit includes the X-ray source 9, the flat panel detector 11, and the cone-beam CT image reconstruction program stored in the computer 17.

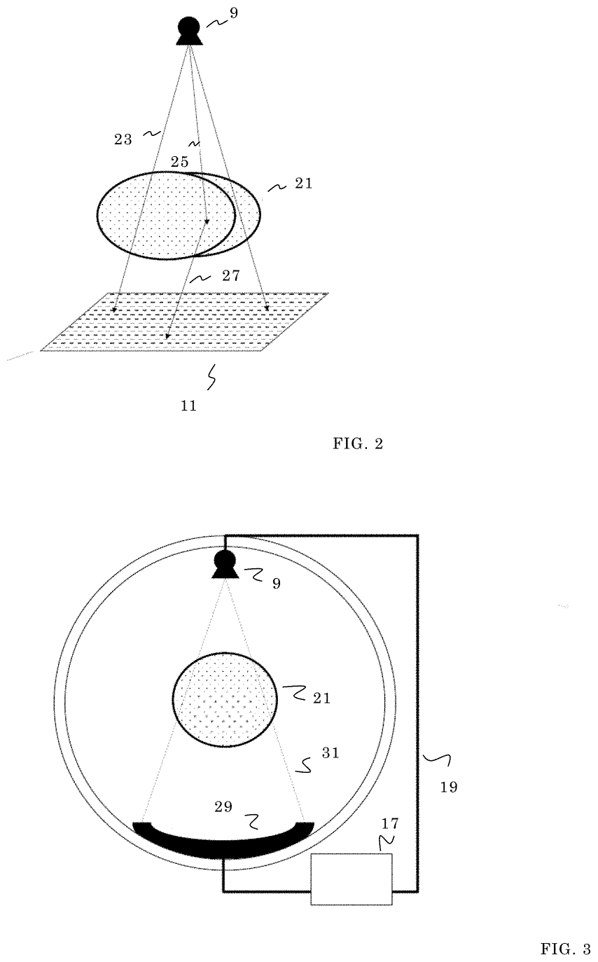

FIG. 2 shows X-ray transport starting at an X-ray source 9, passing through the human body 21, and finally reaching on a flat panel detector 11. The transported X-ray beams are divided into a direct X-ray beam 23 and a scattered X-ray beam 27 that scatters inside the human body 21, both of which reach the flat panel detector 11. An X-ray beam 25 depicts a beam before scattering. It is known that scattered X-ray decreases contrast of projection images.

FIG. 3 illustrates an example diagnostic X-ray CT unit where a fan beam X-ray 31 is emitted from the X-ray source 9. In this case, a couch (not shown) with a human body 21 is continuously translated in the longitudinal direction in order to acquire a large volume of CT images. The X-ray beams reach the detector 29 after passing through the human body 21. The detector 29 consists of a large number of detector elements aligned on an arc. A collimator (not shown) is also placed with each detector element to avoid scattered beam contamination. Control computer 17 and control signal cable 19 are also employed in the same way as shown in FIG. 1. It is possible to separately extract element density images and electron density images from diagnostic X-ray CT images.

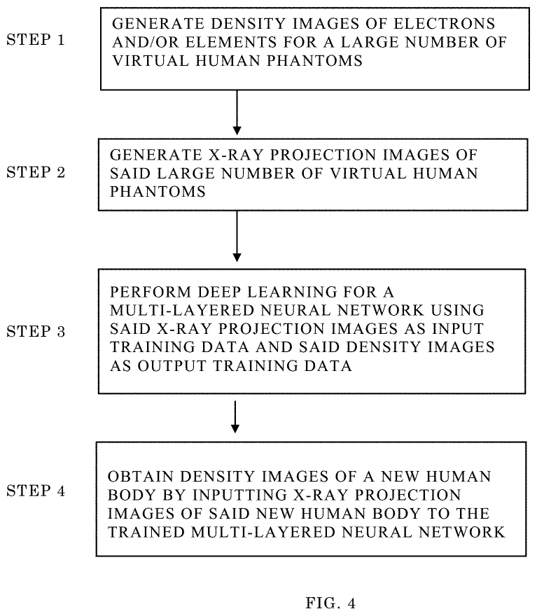

FIG. 4 shows a flowchart that describes a method for obtaining density images of a human body, in other words, scatter-free X-ray cone-beam CT image reconstruction according to the present embodiment. In STEP 1, density images of electrons and/or elements are generated for a large number of virtual human phantoms. In more details, a publication of “Annals of ICRP, ICRP Publication 110, Adult Reference Computational Phantoms, Volume 39, No. 2, 2009, https://www.icrp.org/publication.asp?id=icrp %20publication %20110” contains standard numerical values for male and female bodies, the disclosure of which is hereby incorporated by reference. Standard human body models for males and females are separately generated by referring to this publication. Then statistical models such as Gaussian models are employed, where the above standard numerical values are used as mean values of the Gaussian distributions. In this way, we can randomly sample shape parameters and density distributions of electrons and/or elements, resulting in a large number of different virtual human phantoms in a computer. In STEP 2, X-ray projection images of the large number of virtual human phantoms are generated. In more details, an X-ray projection image is obtained by calculating direct X-ray and scattered X-ray contributions separately using the density distributions and a given X-ray spectrum of the X-ray source. In STEP 3, deep learning is performed for a multi-layered neural network using said x-ray projection images as input training data and said density images of electrons and/or elements as output training data. In STEP 4, density images (electrons and/or elements) of a new human body is obtained by inputting x-ray projection images of said new human body to the trained multi-layered neural network. More detailed deep learning techniques are disclosed in U.S. Pat. No. 8,504,361B2, the disclosure of which is hereby incorporated by reference. The 8504361 patent shows deep learning for a multi-layered neural network with training input text images and training output label images, in order to recognize a new text image. The deep learning technique employed in the present embodiment is mathematically the same as that shown in the above US patent. Another publication of “Kida S, Nakamoto T, Nakano M, et al. Cone Beam Computed Tomography Image Quality Improvement Using a Deep Convolutional Neural Network. Cureus 10: e2548, 2018. doi: 10.7759/cureus.2548” also shows end to end deep learning, the disclosure of which is hereby incorporated by reference.

FIG. 5 is a diagram that shows a method for calculating a direct X-ray projection image of a virtual human phantom. A direct X-ray beam 23 from the X-ray source 9 passes straight through a virtual human phantom 22 and reaches a flat panel detector 11. Direct X-ray attenuation can be calculated by giving a number of straight trajectories starting at the X-ray source 9. The incident X-ray intensity (observed number of photons) on the i-th detector element 33 of the flat panel detector 11 is given by Equation 1.

$${n}_{i}^{total} = {\sum }_{E} {n}_{i} ( E ) = {\sum }_{E} {n}_{0} ( E ) {e}^{{\sum }_{j} - {a}_{ij} {μ}_{j} ( E )} \qquad Equation 1$$

The Equation 1 indicates that the number of photons, nᵢ(E), reaching the i-th detector element 33 of the flat panel detector 11 decays exponentially from the initial entry value of n₀(E) on the surface of the virtual human phantom 22. Because the number of initial photons is a function of photon energy, E, the photons reaching the flat panel are counted energy by energy, and then accumulated over all the energies. The attenuation within a j-th voxel 35 of the virtual human phantom 22 is governed by an exponentially decayed form of the product of a path length aᵢⱼ and a linear attenuation coefficient μⱼ(E). Multiplying all the voxel contributions leads to the overall attenuation inside the phantom along each straight trajectory as shown in the righthand side of Equation 1.

$${μ}_{j} ( E ) = {\sum }_{m} {w}_{m} {μ}_{mj} ( E ) . \qquad Equation 2$$

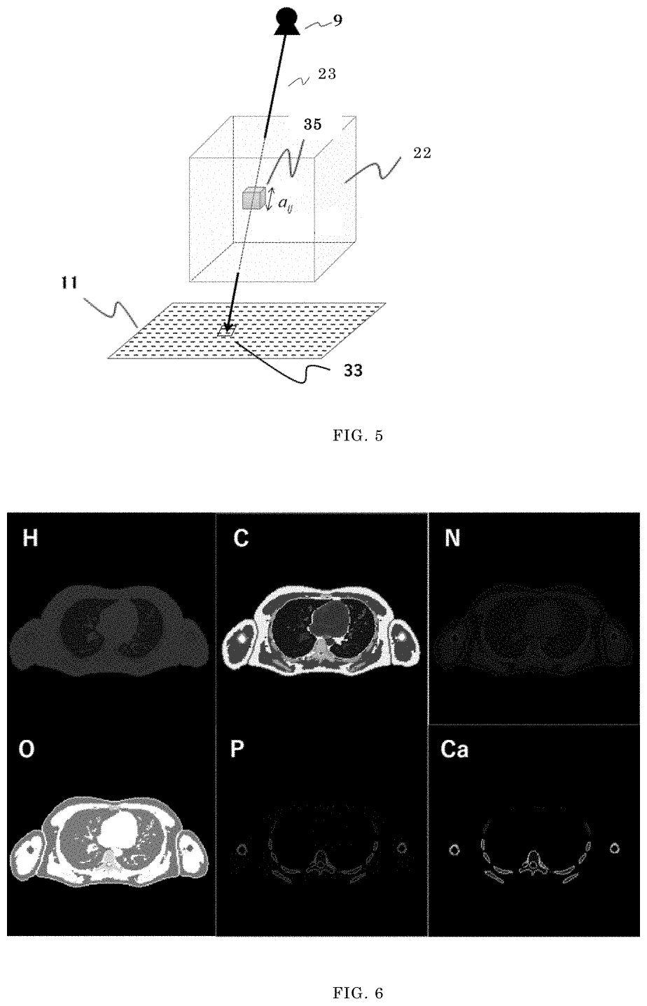

Because a human body has several different elements such as carbon, hydrogen, nitrogen, oxygen etc, the linear attenuation coefficient μⱼ(E) in each voxel needs to consider the elemental composition. The Equation 2 indicates that the linear attenuation coefficient can be calculated by weighted averaging according to each elemental composition ratio wₘ (m=1, 2, . . . ), where the summation of wₘ is normalized to 1. The major elements that constitute a human body are hydrogen (H), carbon (C), nitrogen (N), oxygen (O), phosphorus (P), and calcium (Ca). The elemental composition ratio wₘ differs organ by organ. Aforementioned “Annals of ICRP, ICRP Publication 110” contains elemental composition ratios in a standard human phantom, and FIG. 6 shows calculated distributions of each element listed above on a particular axial cross section by referring to the above ICRP publication. Because linear attenuation coefficients are known element by element, the linear attenuation coefficient in each voxel can be calculated using Equation 2.

FIG. 7 is an example trajectory of scattered X-ray beams 27. The beam 25 was emitted from the X-ray source, and the scattered beam 27 changes the travelling direction within the virtual human phantom 22, reaching a flat detector panel 11. A publication of “Shimomura T, Haga A. Computed tomography image representation using the Legendre polynomial and spherical harmonics functions. Radiol Phys Technol. 14:113-121. 2021. doi: 10.1007/s12194-020-00604-0.” teaches Equation 3 shown below, the disclosure of which is hereby incorporated by reference. The number of scattered photons, D, reaching the i-th element of the flat panel detector 11 can be approximately given by Equation 3, where Y₁ₘ(θ, φ) is a spherical harmonic function, and k₁ₘ(r,r′) is given by Equation 4 under a known scatter kernel of K(r-r′). The scatter kernel can be one described in a publication of “Harry R. Ingleby, Idris A. Elbakri, Daniel W. Rickey, Stephen Pistorius, Analytical scatter estimation for cone-beam computed tomography, Proc. SPIE 7258, Medical Imaging: Physics of Medical Imaging, 725839, 2009. doi: 10.1117/12.813804”, the disclosure of which is hereby incorporated by reference. The scattering kernel in this case is based on a Klein-Nishina formula. R₁ₘ(r) is a coefficient given by Equation 5, when voxel value f(r,θ,φ) of the virtual human phantom is represented by spherical harmonics.

$$\begin{matrix}D ( \vec{r} ) \text{\textasciitilde} {\sum }_{lm} {Y}_{lm} ( θ , ϕ ) \int {k}_{lm} ( r , {r}^{′} ) {R}_{lm} ( r , {r}^{′} ) {R}_{lm} ( {r}^{′} ) {dr}^{′} & Equation 3 \\ {k}_{lm} ( r , {r}^{′} ) = \int K ( r - {r}^{′} ) {Y}_{lm} ( θ , ϕ ) {Y}_{lm}^{*} ( {θ}^{′} , {ϕ}^{′} ) d \hat{r} d {\hat{r}}^{′} & Equation 4 \\ {R}_{lm} ( r ) = {\int }_{0}^{2 π} d φ {\int }_{0}^{π} sin θ d θ f ( r , θ , φ ) {Y}_{lm} ( θ , φ ) & Equation 5\end{matrix}$$

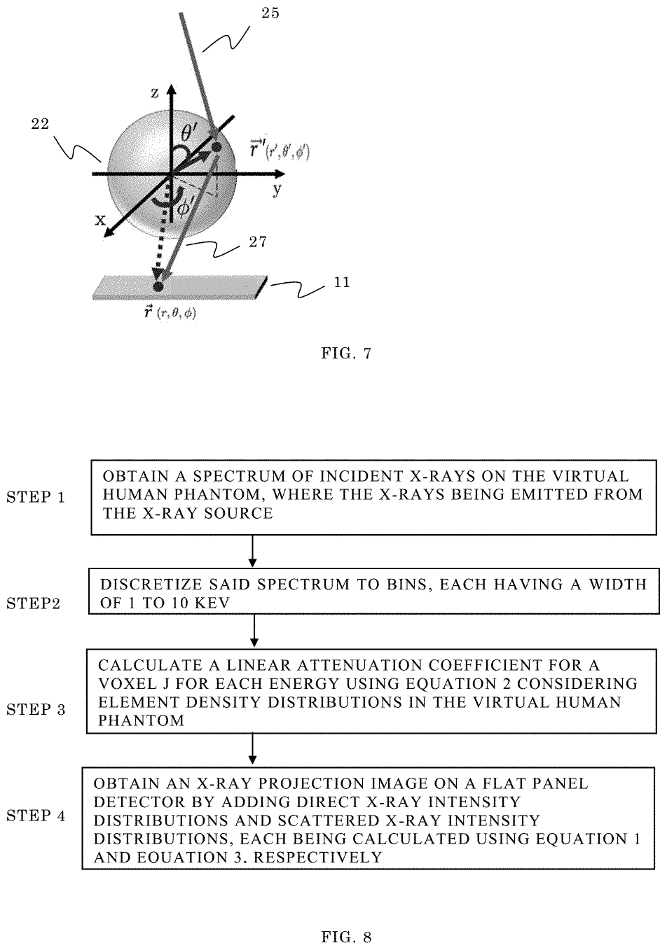

FIG. 8 shows a flowchart for calculating X-ray projection images, which is executed by a program stored in the computer 17 shown in FIG. 1. In STEP 1, a spectrum of incident x-rays on the virtual human phantom is obtained, where the x-rays being emitted from the x-ray source. A spectrum in this context is the number of photons as a function of the photon energies. Methods for estimating the spectrum is already known. For example, a publication of “Hasegawa Y, Haga A, Sakata D, Kanazawa Y, Tominaga M, Sasaki M, Imae T, Nakagawa K, Estimation of X-ray Energy Spectrum of Cone-Beam Computed Tomography Scanner Using Percentage Depth Dose Measurements and Machine Learning Approach. Journal of the Physical Society of Japan, 90, 074801, 2021; doi:10.7566/JPSJ.90.074801” discloses a method, the disclosure of which is hereby incorporated by reference, where a large number of combination of depth doses and x-ray spectrums are generated by Monte Carlo calculation under various experimental conditions, and then those data are fed into a multi-layered neural network for deep learning. After the learning is completed, inputting a newly measured depth dose to the trained neural network results in a corresponding X-ray spectrum, the disclosure of which is hereby incorporated by reference. Another method shown in this publication is an iterative estimation whereby the measured depth dose is approximated by a linear sum of various depth doses resulting from a number of different monoenergy X-ray beams. By minimizing the difference between the measured and calculated depth doses, an X-ray spectrum is obtained as weights of the monoenergy X-ay beams. On the other hand, a publication of “Liu B, Yang H, Lv H, Li L, Gao X, Zhu J, Jing F, A method of X-ray source spectrum estimation from transmission measurements based on compressed sensing, Nuclear Engineering and Technology, 52, 1495-1502, 2020. doi:10.1016/j.net.2019.12.004” discloses another method, the disclosure of which is hereby incorporated by reference, whereby X-ray beams from the X-ray source is irradiated to metal phantoms having various thicknesses and materials. By measuring the exit dose, the number of photons is estimated as a function of the X-ray energy. In the present embodiment, any one of the known methods described above can be used to estimate the X-ray spectrum. In STEP 2, said spectrum is discretized to bins, each having a width of 1 to 10 keV (kilo electron volt) for further calculation. In STEP 3, a linear attenuation coefficient for a voxel j is calculated for each energy using the Equation 2 considering element density distributions in the virtual human phantom. In STEP 4, an X-ray projection image on a flat panel detector is calculated by adding direct x-ray intensity distributions and scattered x-ray intensity distributions, each being calculated using Equation 1 and Equation 3, respectively. In reality, the flat panel detector has noise components due to dark currents, and therefore it is preferable to add this noise amount by measurement.

FIG. 8 presumes cone-beam CT unit shown in FIG. 1 and FIG. 2. For diagnostic CT unit shown in FIG. 3, the X-ray beams are usually fan beams with a possible single row detector placement and in this case voxels should be replaced with pixels throughout this patent application.

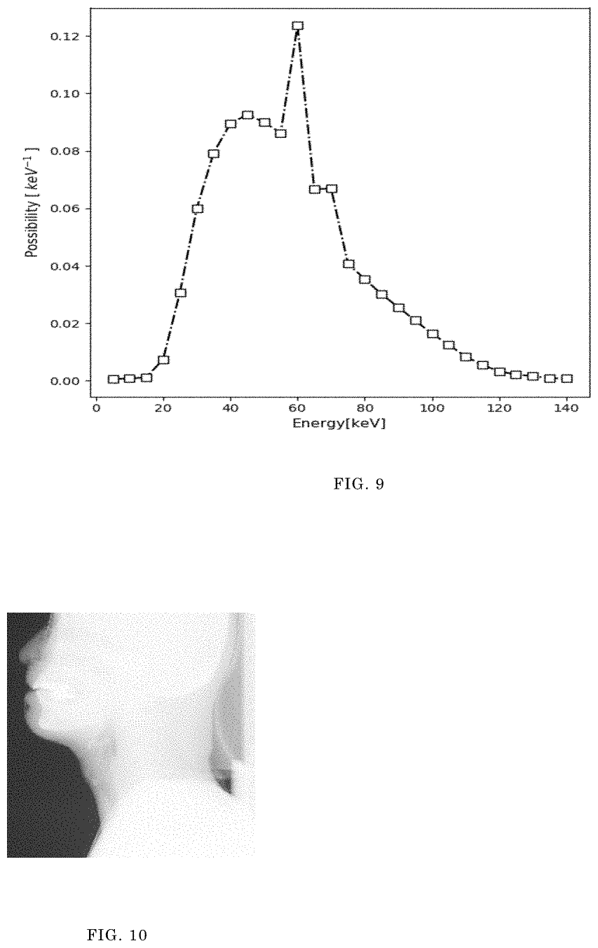

FIG. 9 is an example X-ray spectrum obtained in the STEP 1 of FIG. 8, and FIG. 10 is an example projection image of a virtual human phantom calculated by the flowchart in FIG. 8.

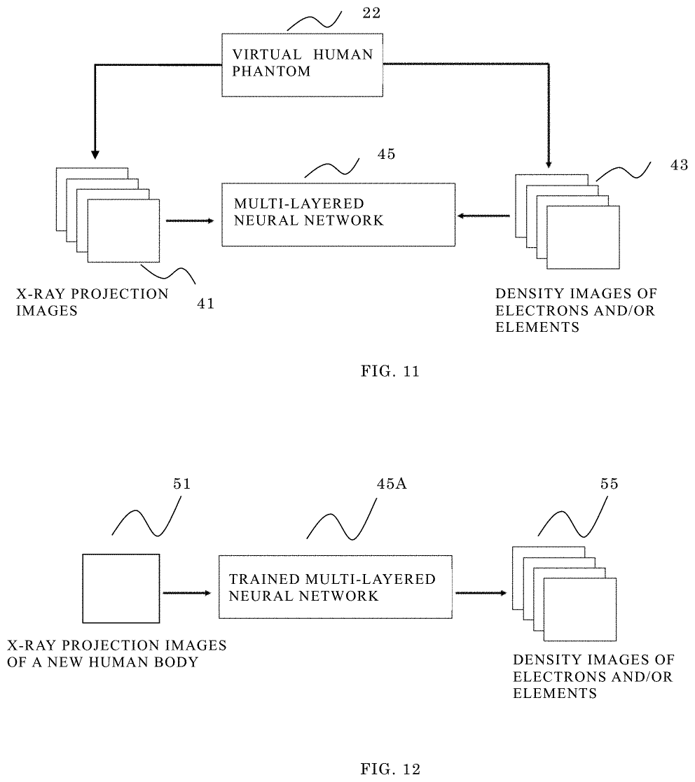

FIG. 11 is a block diagram for performing STEP 1 through STEP 3 of FIG. 4. Density images of electrons and/or elements are generated for a large number of virtual human phantoms 22 having various shapes and material distributions. X-ray projection images 41 of the large number of virtual human phantoms 22 are also generated. Based on these image data, deep learning is performed for a multi-layered neural network 45 using said x-ray projection images as input training data and said density images of electrons and/or elements 43 as output training data.

FIG. 12 is a block diagram for performing STEP 4 of FIG. 4. By inputting X-ray projection images 51 of a new human body acquired by a cone-beam CT unit to the trained multi-layered neural network 45A, density images 55 of electrons and/or elements of the new human body can be obtained. As a result, X-ray projection image 51 having scattered components is converted to density images 55 of electrons and/or elements. In this embodiment, projection images are employed as input data to the neural network, but it is also possible to use a cone-beam CT image instead of the projection images, which will be described as the next embodiment.

### Detailed Description: Second Embodiment with FIGS. 13-17

FIG. 13 is another flowchart that describes an X-ray cone-beam CT image reconstruction without having scattering components. Differences from FIG. 4 are 1) cone-beam CT images are reconstructed in STEP 3 after obtaining projection images, 2) the cone-beam CT images are inputted to a neural network as input training data for deep learning in STEP 4, and 3) a cone-beam CT image of a new human body is inputted to the trained neural network in STEP 5. Others are the same as those described in FIG. 4.

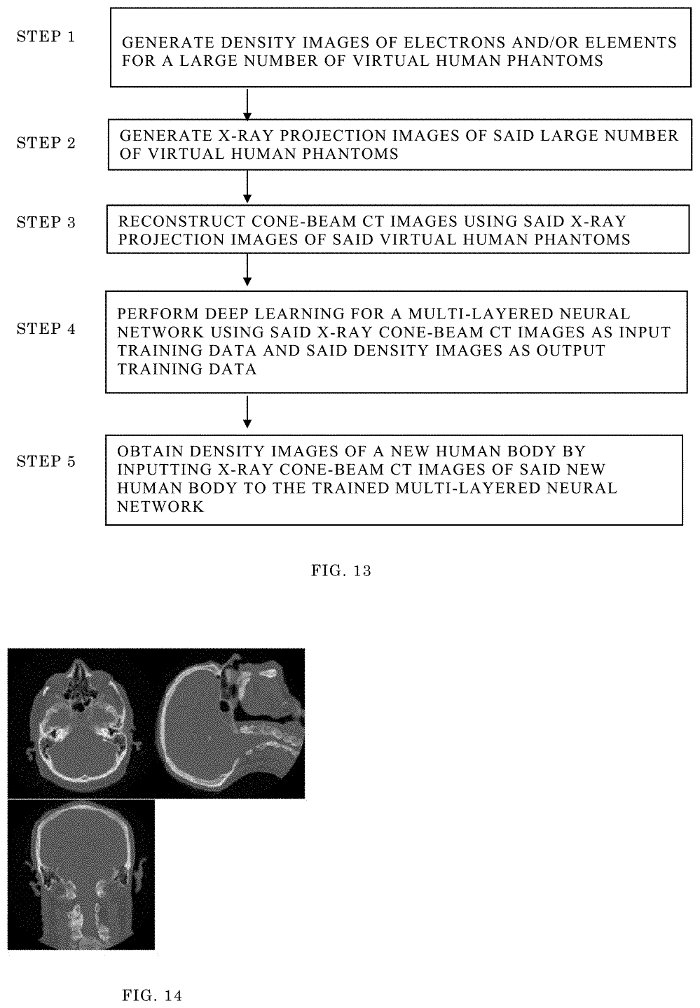

FIG. 14 is an example cone-beam CT image reconstructed by STEP 3 of FIG. 13, with three orthogonal views.

FIG. 15 is a block diagram that shows a deep learning process of a multi-layered neural network 49 with input training data of cone-beam CT images 47 and output training data of density images 43. FIG. 15 is the same as FIG. 11 except that cone-beam CT images 47 are used as input training data. As was mentioned in the background section, Feldkamp's back projection method is employed in the STEP 3 of FIG. 13 as well as in FIG. 15 for reconstructing the cone-beam CT image 47.

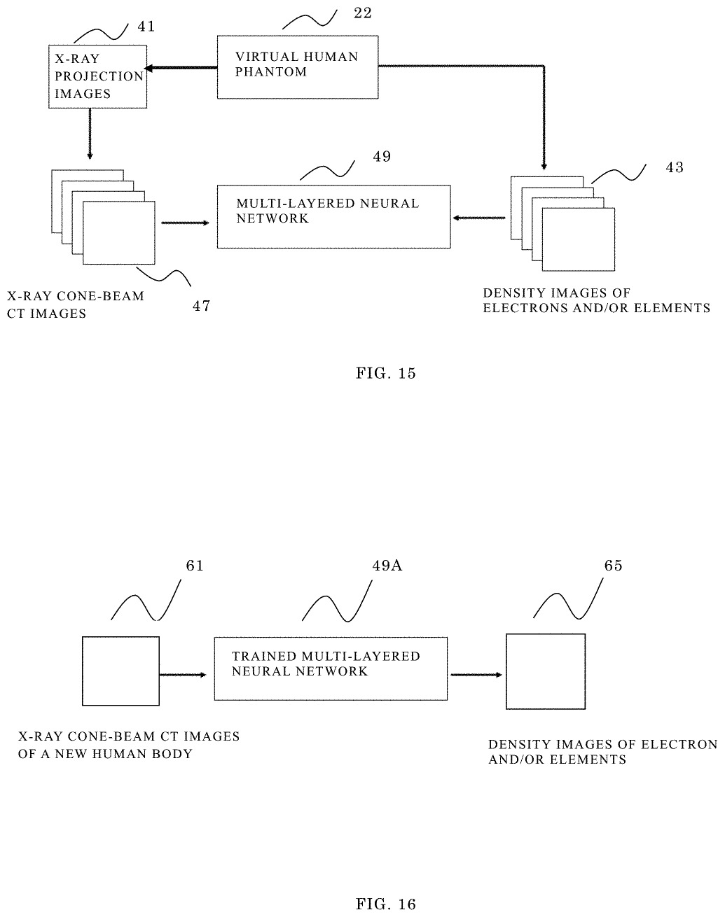

FIG. 16 is a block diagram showing that a cone-beam CT image 61 of a new human body is fed into the trained neural network 49A, leading to density images 65 of electrons and/or elements of the same human. The difference from FIG. 12 is that a cone-beam CT image 61 is inputted to the trained neural network 49A.

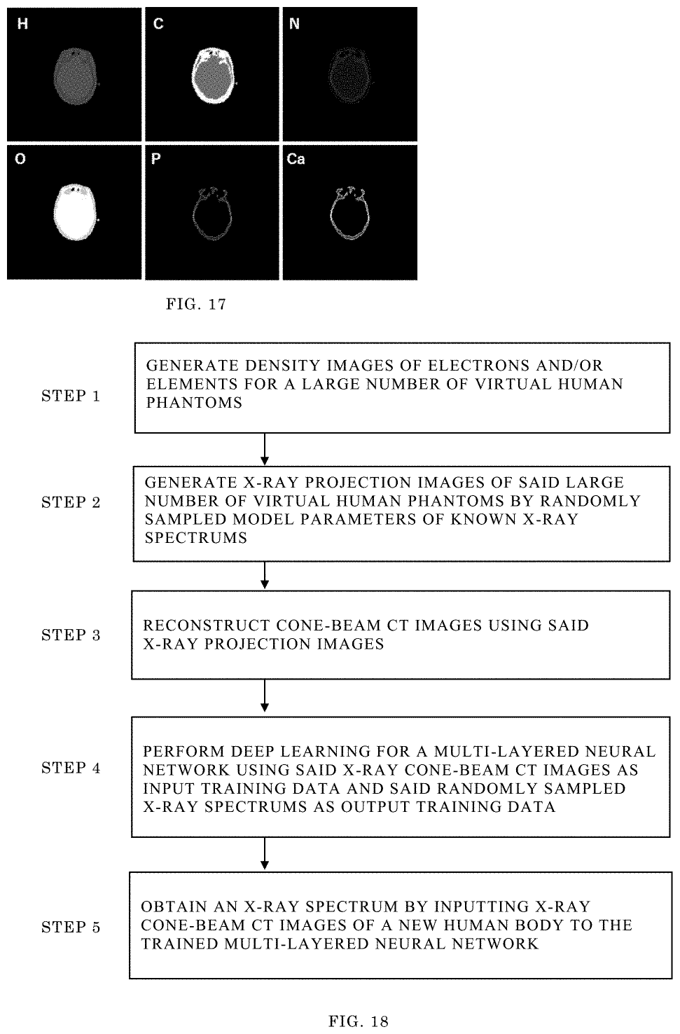

FIG. 17 is example density images of the elements in the human body as an output from the trained multi-layered neural network shown in FIG. 16, consisting of hydrogen (H). carbon (C), nitrogen (N), oxygen (O), phosphorus (P), and calcium (Ca). An electron density image can be calculated by summation of product of each element density image and Z/A, where Z denotes atomic number and A denotes atomic weight.

### Detailed Description: Third Embodiment with FIGS. 18-20

As was mentioned, the first embodiment employed several known methods to obtain the X-ray spectrum of the X-ray source in STEP 1 of FIG. 8. In this embodiment, a new method for obtaining the X-ray spectrum is described using deep learning.

FIG. 18 is a flowchart showing a new method for obtaining an X-ray spectrum of the X-ray source 9. In STEP 1, density images of electrons and/or elements for a large number of virtual human phantoms are generated. In STEP 2, X-ray projection images of said large number of virtual human phantoms are generated by randomly sampled model parameters of known X-ray spectrums. In STEP 3, cone-beam CT images are calculated using said X-ray projection images. In STEP 4, deep learning for a multi-layered neural network is performed using said X-ray cone-beam CT images as input training data and said randomly sampled X-ray spectrums as output training data. In STEP 5, an X-ray spectrum is obtained by inputting X-ray cone-beam CT images of a new human body to the trained multi-layered neural network.

Based on previously measured X-ray spectrums having different anode-cathode voltages, a plurality of parametric models of the measured X-ray spectrums are created as s plurality of standard models. Then the model parameters are statistically varied to generate a large number of different X-ray spectrum data. A large number of projection data can be generated by using the large number of X-ray spectrums, which is how the projection images are generated in STEP 2 of this embodiment.

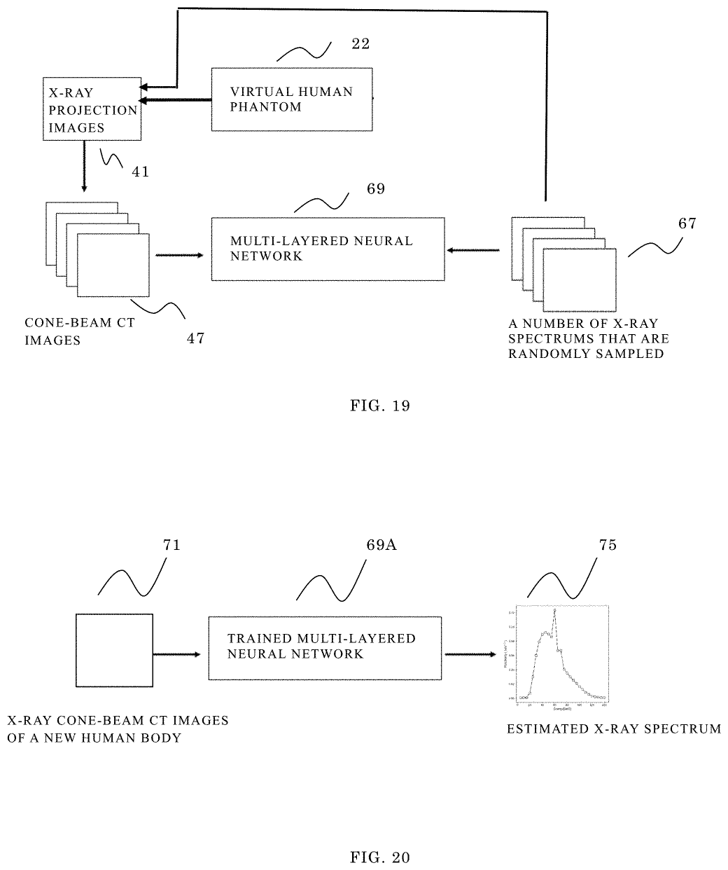

FIG. 19 is a block diagram that shows a deep learning process from STEP 1 through STEP 4 of FIG. 18, where a multi-layered neural network 69 is trained by cone-beam CT images 47 as input data and X-ray spectrums 67 as output data. The cone-beam CT images 47 is reconstructed by projection images 41, which is calculated using the randomly sampled X-ray spectrum. As was mentioned earlier, the cone-beam CT reconstruction in STEP 3 of FIG. 18 and in FIG. 19 can be performed by Feldkamp's back projection method.

FIG. 20 is a block diagram that provides an X-ray spectrum as an output of a trained neural network 69A, as shown in the 5 of FIG. 18, after inputting a cone-beam CT image 71 of a new human body. This is meaningful when the cone-beam CT image came from a different institution and no X-ray spectrum information is available. When the X-ray spectrum is unknown, projection images cannot be calculated for deep learning. Using the present embodiment, an X-ray spectrum is obtained by inputting the cone-beam CT image.

In this embodiment, cone-beam CT images are used as input training data for deep learning. It is also possible to use projection images as input training data for the deep learning, which are available immediately before reconstructing the cone-beam CT image. In this latter case, inputting projection images of a new human body to the trained neural network results in a corresponding X-ray spectrum.

In this embodiment, a plurality of standard X-ray spectrum models are determined and then a large number of X-ray spectrums are generated by randomly changing the model parameters, according to Gaussian distribution statistics for example. The resulting large number of X-ray spectrums are used to generate a large number of projection images. By referring to FIG. 4 and FIG. 13, it is possible to train a multi-layered neural network with projection images or cone-beam CT images of virtual human phantoms as input training data, and density images of electrons and/or elements as output training data. The advantage of this procedure is that deep learning can be performed without knowing the X-ray spectrum of the X-ray source. In other words, density images of electrons and/or elements of a new human body can be estimated without knowing the X-ray spectrum.

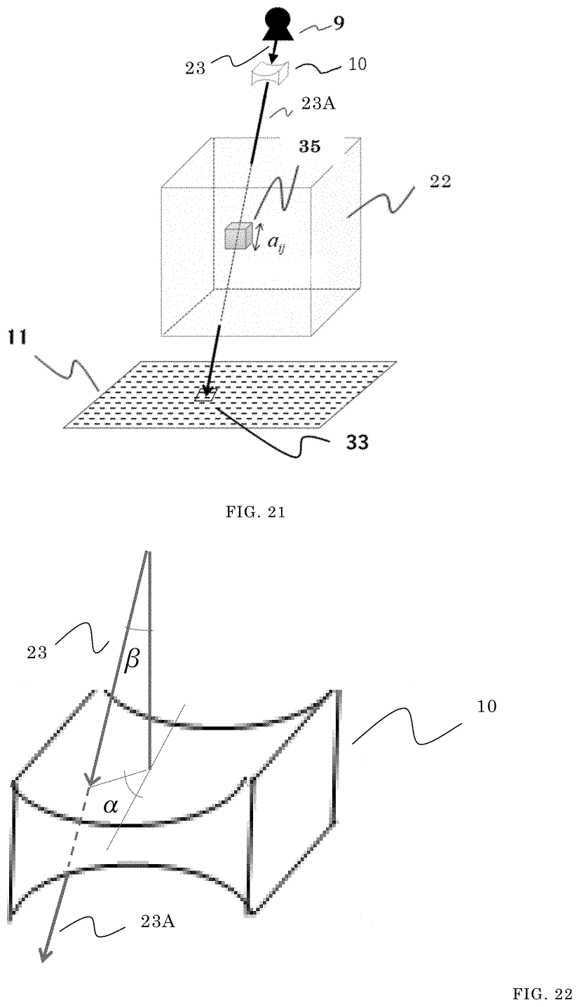

### Detailed Description: Fourth Embodiment with FIGS. 21-25

Most of the cone-beam CT unit has a metal-made bowtie filter placed in the proximity of the X-ray source to improve image quality.

FIG. 21 is a diagram showing a method for calculating a direct X-ray projection image of a virtual human phantom with a bowtie filter 10 placed near the X-ray source 9, where direct X-ray beams 23 and 23A correspond to the beams before and after passing the bowtie filter 10, respectively. Those two beams have different X-ray intensities and different X-ray spectrums, thus requiring corrections.

FIG. 22 shows a perspective view of a typical metal-made bowtie filter 10, having a thicker beam path length at both sides. The bowtie filter serves to improve the image quality of the cone-beam CT by decreasing X-ray intensity at both left and right sides of the human body having a thinner beam path length. This can avoid signal saturation of the flat panel detector 11. The X-ray intensities of beams 23 and 23A are given by Equation 6.

$${I}_{2} ( α , β , E ) = {e}^{- μ ( E ) d ( α , β )} {I}_{1} ( E ) \qquad Equation 6$$

Iᵢ(E) is an intensity of beam 23 as a function of energy E, and which is therefore an energy spectrum of beam 23, whereas I₂(α, β, E) is an energy spectrum of beam 23A, where E is an energy of X-ray beams. The X-ray attenuation relates to the beam path length inside the bowtie filter 10. The beam path length is related to cone angle α and β, which is denoted as d(α, β). In addition, attenuation per length in the metal bowtie filter depends on material property and X-ray energy, thus denoted as μ(E), leading to a total attenuation of exp {−μ(E)d(α, β)}, which is given as a correction factor in Equation 6. Using this Equaton 6, the X-ray spectrum of beams 23A can be calculated from the X-ray spectrum of beams 23.

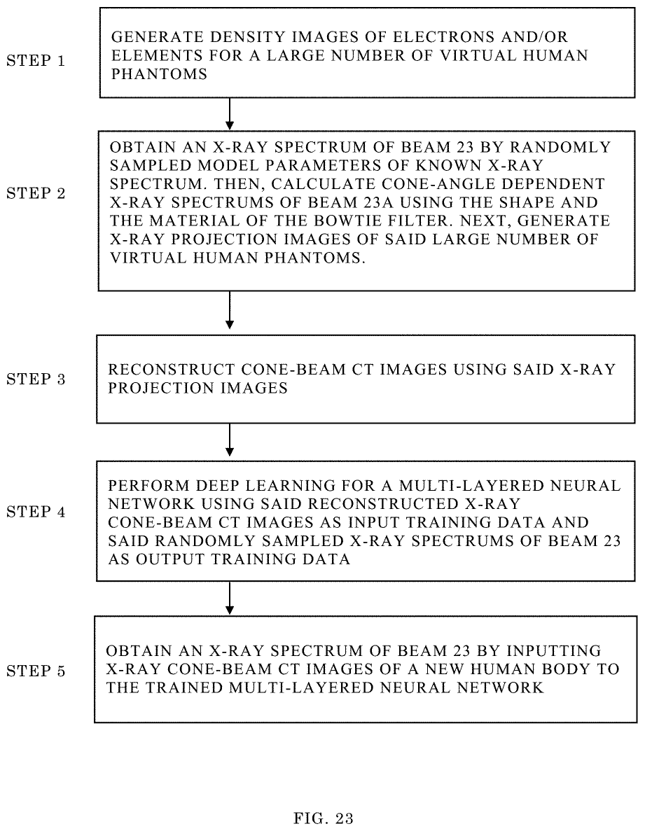

FIG. 23 is a flowchart that shows a method for calculating an X-ray spectrum when a bowtie filter is placed with known shape and material information. The flowchart shown in FIG. 23 is similar to that in FIG. 18 except STEP 2 because of additional bowtie filter. In STEP 1, density images of electrons and/or elements are generated for a large number of virtual human phantoms. In STEP 2, an X-ray spectrum of beam 23 is given by randomly sampled model parameters of known x-ray spectrum of the X-ray source without the bowtie filter. Then, cone-angle dependent X-ray spectrum of beam 23A is calculated by Equation 6, using the shape and the material of the bowtie filter. Next, X-ray projection images of said large number of virtual human phantoms are generated. In STEP 3, cone-beam CT images are generated using said x-ray projection images. In STEP 4, deep learning is performed for a multi-layered neural network using said reconstructed X-ray cone-beam CT images as input training data and said randomly sampled X-ray spectrums of beam 23 as output training data. In STEP 5, an X-ray spectrum of beam 23 is obtained by inputting an X-ray cone-beam CT image of a new human body to the trained multi-layered neural network.

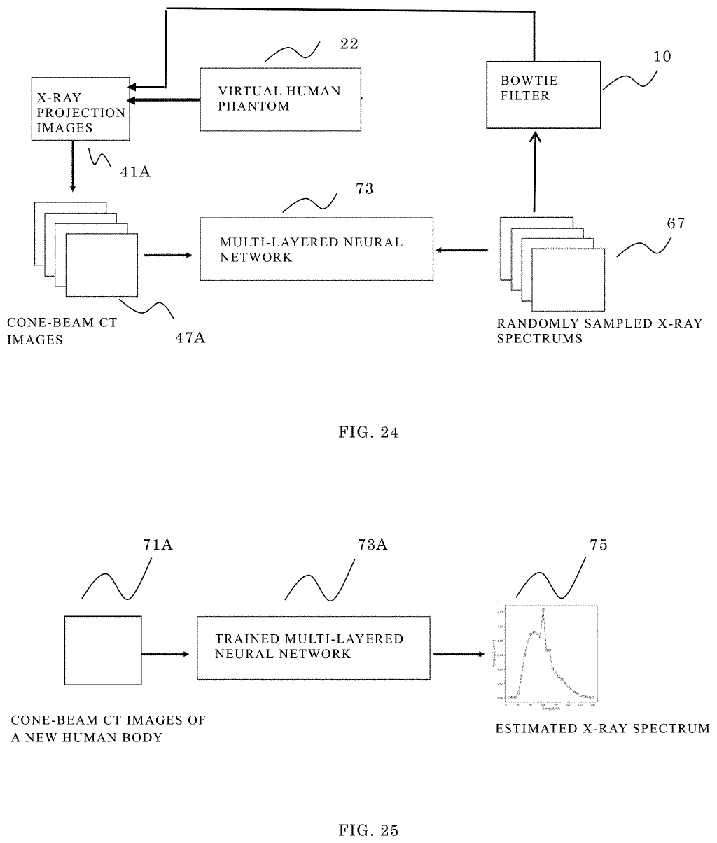

FIG. 24 is a block diagram of a deep learning process shown as STEP 1 through STEP 4 of FIG. 23. The multi-layered neural network 73 is trained by cone-beam CT images 47A as input data and X-ray spectrums 67 as output data. Cone-beam CT images 47A are obtained from projection images 41A of virtual human phantom 22 using aforementioned Feldkamp's back projection method. To calculate projection images 41A, randomly sampled X-ray spectrum 67 of the X-ray source was converted to the X-ray spectrum after passing the bowtie filter 10. Others are the same as shown in FIG. 19.

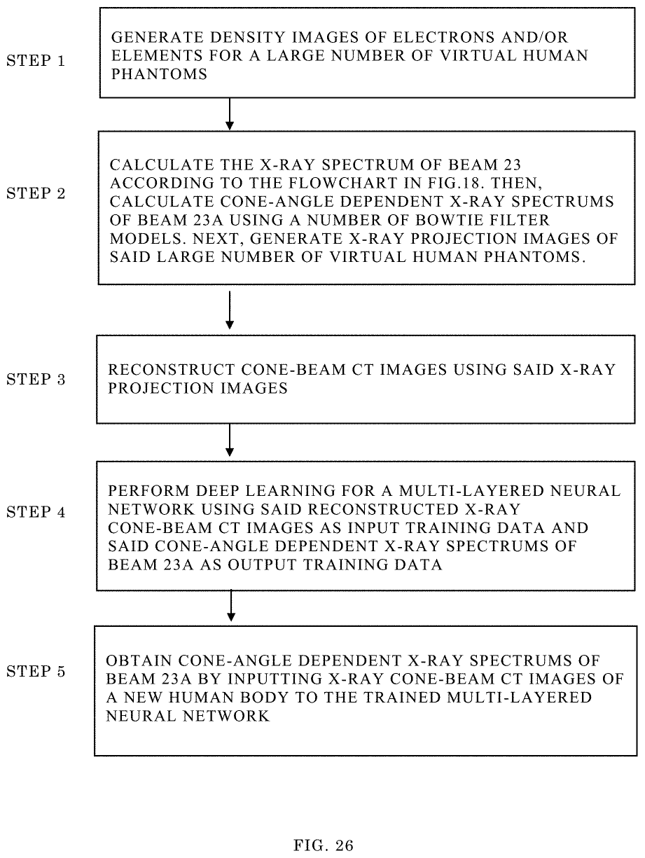

FIG. 25 is a block diagram for obtaining an X-ray spectrum by inputting a bowtie-filtered cone-beam CT image 71A of a new human body to a trained neural network 73A as shown in STEP 5 of FIG. 23. This is meaningful when the cone-beam CT image came from a different institution and no X-ray spectrum information is available. When the X-ray spectrum is unknown, projection images cannot be calculated for deep learning. Using the present embodiment, an X-ray spectrum is obtained by inputting the cone-beam CT image with known bowtie filter information but without knowing X-ray spectrum information.

In this embodiment, cone-beam CT images are used as input training data for deep learning. It is also possible to use projection images as input training data for the deep learning, which are available immediately before reconstructing the cone-beam CT image. In this latter case, inputting projection images for a new human body to the trained neural network results in an X-ray spectrum.

Another variation is that cone-angle dependent X-ray spectrums after passing the bowtie filter are used as output training data. In this case, inputting projection images or cone-beam CT image of a new human body results in cone-angle dependent X-ray spectrums after passing the bowtie filter as output from the trained multi-layered neural network.

### Detailed Description: Fifth Embodiment with FIGS. 26-28

FIG. 26 is a flowchart that shows a method for calculating an X-ray spectrum when a bowtie filter without shape and material information is placed. In STEP 1, density images of electrons and/or elements are generated for a large number of virtual human phantoms having different geometric and density parameters. In STEP 2, the X-ray spectrum of beam 23 is calculated according to the flowchart in FIG. 18. Then, cone-angle dependent X-ray spectrums of beam 23A are calculated using a number of bowtie filter models having different shapes and/or materials. Next, X-ray projection images of said large number of virtual human phantoms are calculated. In STEP 3, cone-beam CT images are reconstructed using said x-ray projection images. In STEP 4, deep learning is performed for a multi-layered neural network using said reconstructed X-ray cone-beam CT images as input training data and said cone-angle dependent X-ray spectrums of beam 23A as output training data. In STEP 5, cone-angle dependent X-ray spectrums of beam 23A are obtained by inputting X-ray cone-beam CT images of a new human body to the trained multi-layered neural network. The flowchart shown in FIG. 26 is similar to that in FIG. 23, but in this embodiment, STEP 2 is significantly different because shape and/or material information of the bowtie filter is unknown.

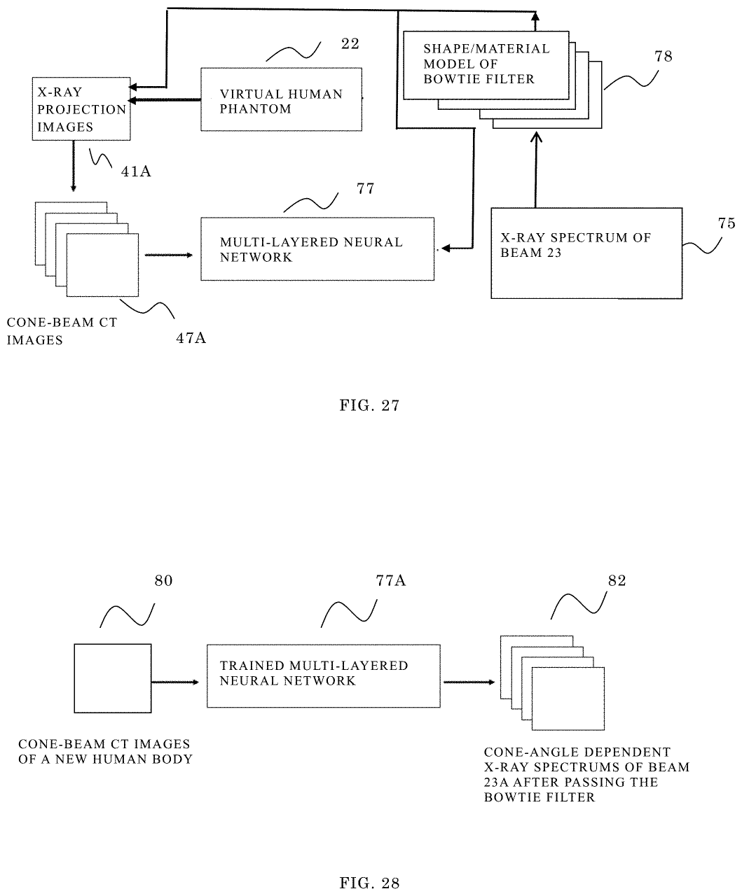

FIG. 27 is a block diagram of a deep learning process shown as STEP 1 through STEP 4 of FIG. 26. The X-ray spectrum 75 of beam 23 is calculated according to FIG. 18 for example. Subsequently, using a large number of bowtie filter models 78 with varied shape and material properties, a large number of cone-angle dependent X-ray spectrums of beam 23A are calculated. Then X-ray projection images 41A of a large number of virtual human phantoms 22 are calculated, leading to cone-beam CT images 47A. Deep learning of a multi-layered neural network 77 is conducted using cone-beam CT images 47A as input training data, and cone-angle dependent X-ray spectrums of beam 23A calculated from said large number of bowtie filter models 78 as output training data.

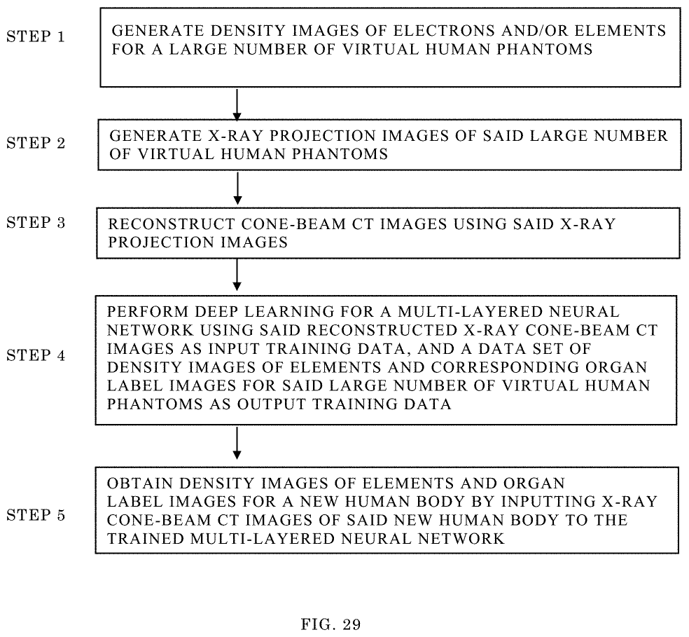

FIG. 28 is a block diagram for obtaining a cone-angle dependent X-ray spectrum of the beam 23A after passing the bowtie filter 10 with unknown shape and/or material information. By inputting a cone-beam CT image 80 of a new human body to the trained multi-layered neural network 77A as shown in STEP 5 of FIG. 26, corresponding cone-angle dependent X-ray spectrums of the beam 23A are obtained. This embodiment is meaningful when the cone-beam CT image came from a different institution and no X-ray spectrum information is available. When X-ray spectrum is unknown, projection images cannot be calculated. Using the present embodiment, cone-angle dependent X-ray spectrums are obtained by inputting a bowtie-filtered cone-beam CT image of a new human body without knowing X-ray spectrum information.

In this embodiment, cone-beam CT images are employed for input training data for deep learning, but projection images immediately before cone-beam CT reconstruction may also be used as input training data. In this case, inputting projection images of a new human body to a trained neural network results in cone-angle dependent X-ray spectrums after passing the bowtie filter.

### Detailed Description: Sixth Embodiment with FIGS. 29-31

FIG. 29 is a flowchart showing a method for calculating a density image with an organ label image by calculating cone-beam CT images of a large number of virtual human phantoms. In STEP 1, density images of electrons and/or elements for a large number of virtual human phantoms are generated. In STEP 2, X-ray projection images of said large number of virtual human phantoms are calculated based on the procedure described in aforementioned embodiments. In STEP 3, cone-beam CT images are obtained using said X-ray projection images preferably by Feldkamp's back projection method that is previously explained. In STEP 4, deep learning is performed for a multi-layered neural network using said reconstructed X-ray cone-beam CT images as input training data, and a data set of density images of elements and corresponding organ label images for said large number of virtual human phantoms as output training data. In STEP 5, a set of density images of elements and corresponding organ label images for a new human body is obtained by inputting X-ray cone-beam CT images of said new human body to the trained multi-layered neural network. It is also possible to calculate electron density images from element density images as mentioned earlier.

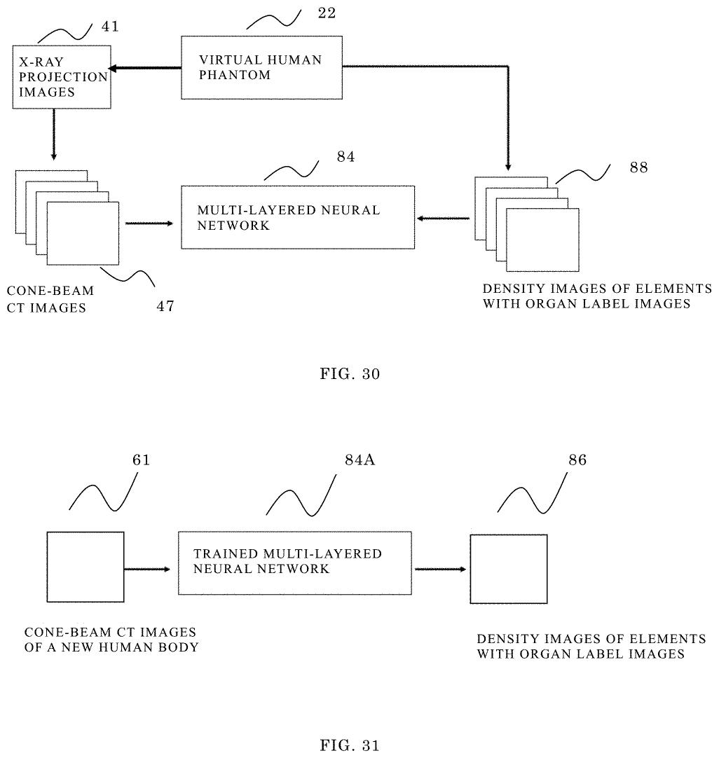

FIG. 30 is a block diagram of a deep learning process shown as STEP 1 through STEP 4 of FIG. 29. A large number of projection images 41 are calculated from a large number of virtual human phantom 22 as described in the previous embodiments. Cone-beam CT images 47 are reconstructed as described in the previous embodiments, the cone-beam CT images 47 being used as input training data. Using a number of virtual human phantoms 22, a set of element density images as well as corresponding organ label images 88 is calculated as output training data. Deep learning for a multi-layered neural network 84 is performed using said input and output training data.

FIG. 31 is a block diagram to realize the calculation process for the STEP 5 of FIG. 29. The trained neural network 84A accepts cone-beam CT image 61 of a new human body as input data, leading to a set of element density images and a corresponding organ label image 86 as output data.

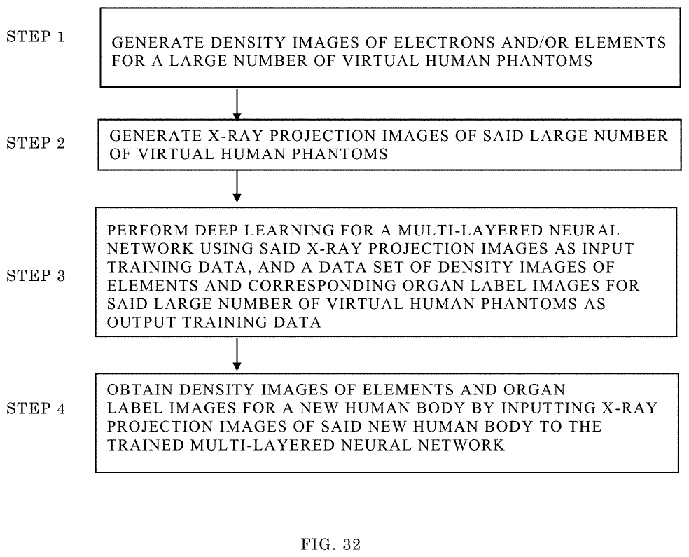

### Detailed Description: Seventh Embodiment with FIGS. 32-36

FIG. 32 is a flowchart for calculating a set of density images and an organ label image based on projection images of a large number of virtual human phantoms. The difference from FIG. 29 is that projection images are employed in STEP 3 as input training data for a neural network. As a result of this change, in STEP 4, the input to the trained network is projection images of a new human body, not cone-beam CT images of the new human body.

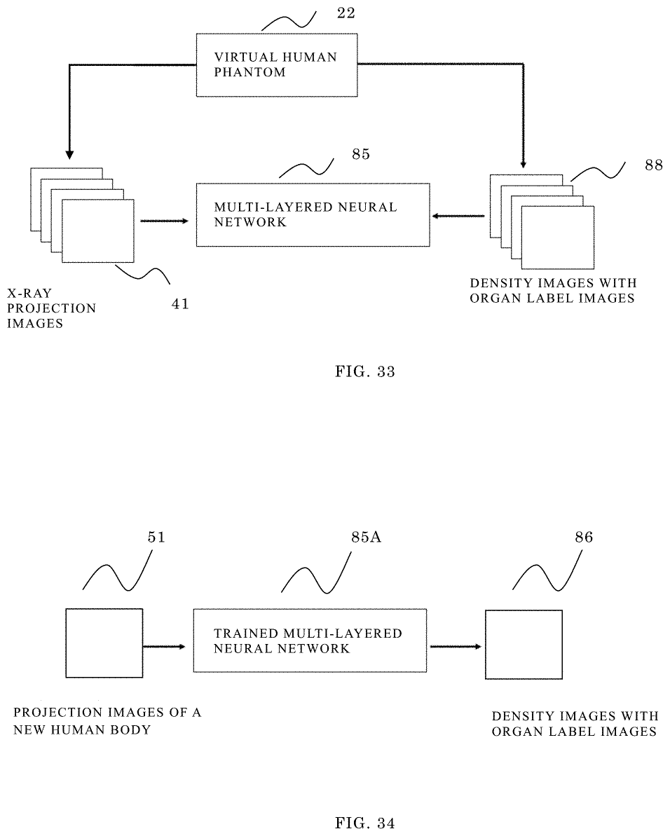

FIG. 33 is a block diagram of a deep learning process as shown in STEP 1 through STEP 3 of FIG. 32. For a large number of virtual human phantoms 22, a large number of projection images 41 are calculated as input training data. Simultaneously, as output training data, a large number of sets of element density images and corresponding organ label images 88 are calculated from the virtual human phantom 22. Subsequently, deep learning of a multi-layered neural network 85 is conducted using said input and output training data.

FIG. 34 is a block diagram showing a calculation process of STEP 4 in FIG. 32. By inputting projection images 51 to the trained neural network 85A after the deep learning in FIG. 33 is completed, a set of element density images and an organ label image 86 is obtained as an output from the trained neural network 85A.

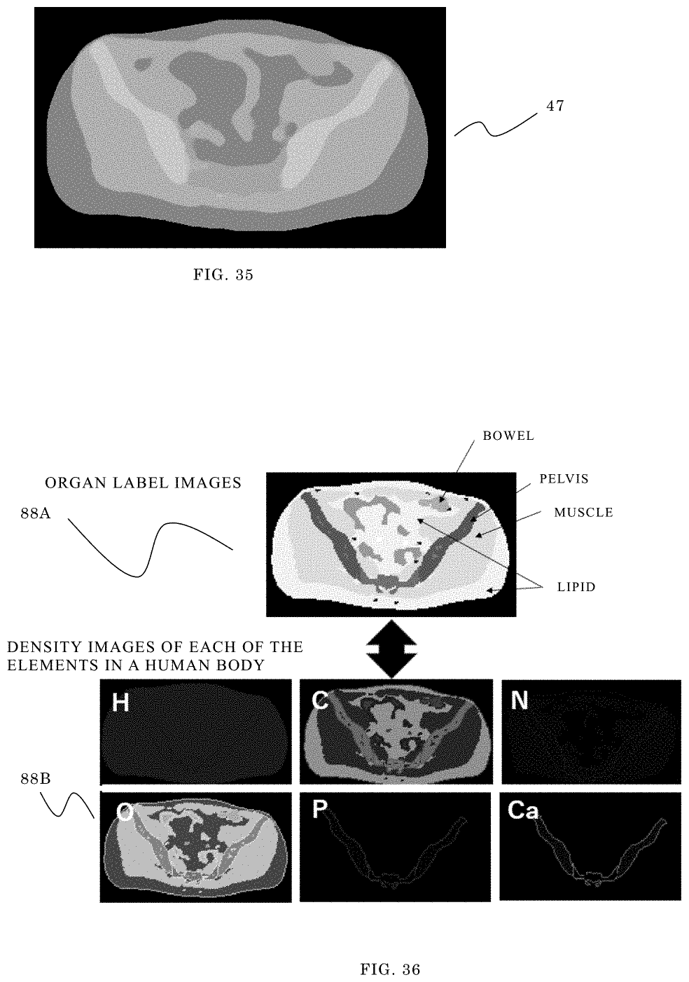

FIG. 35 is an example cone-beam CT image 47 of FIG. 30, which shows an axial slice image at a particular cross section.

FIG. 36 is an example element density images 88B with a corresponding organ label image 88A used for deep learning shown in FIG. 30 or FIG. 33, where the images 88A and 88B correspond to the image set 88 described in FIG. 30 and FIG. 33. In each voxel of the organ label image, one of the organ or tissue names is assigned as a label.

The present invention is not limited to the above-described embodiments, and of course various configurations can be obtained without deviating from the gist of the present invention. For example, cone-beam CT images are mostly referred to in this invention but images from diagnostic CT unit shown in FIG. 3 are also covered by this invention, and thus the present invention can be applied to the diagnostic CT images as well.

In this disclosure, an expression of A and/or B is frequently used, which means “A but not B”, “B but not A”, and “A and B” unless otherwise indicated.

Lastly, the scope of the embodiments should be determined by the appended claims and their legal equivalents, rather than by the examples given.

What is claimed is:

1. A method for calculating density images in a human body, comprising:
(a) generating density images of electrons and/or elements for a large number of virtual human phantoms,
(b) calculating X-ray projection images of said large number of virtual human phantoms,
(c) performing deep learning for a multi-layered neural network using said X-ray projection images as input training data and said density images as output training data,
(d) obtaining density images of a new human body by inputting X-ray projection images of said new human body to the trained multi-layered neural network.

2. The method according to claim 1, wherein the step (b) further comprising:
(b1) determining an X-ray standard spectrum model of an X-ray source, and generating a large number of X-ray spectrums by varying the parameters of said X-ray standard spectrum model,
(b2) discretizing each of said large number of X-ray spectrums,
(b3) calculating a direct X-ray intensity and a scattering process within said virtual human phantom under each energy of said discretized X-ray spectrum with density information of elements and/or electrons,
(b4) adding at least direct X-ray and scattered X-ray intensities for each X-ray energy on each detector of the X-ray flat panel, and then obtaining an X-ray projection image by performing weighted summation for all the X-ray energies according to the spectrum intensity as a function of X-ray energies.

3. The method according to claim 2, wherein the step (b1) is initially conducted without placing a bowtie filter near the X-ray source, and then said generated large number of X-ray spectrums are further adjusted by the shape and material information of a bowtie filter placed near the X-ray source, thereby generating bowtie-filtered cone-angle dependent X-ray spectrums on a virtual human phantom.

4. The method according to claim 1, wherein the step (b) further comprising:
(b1) calculating an X-ray spectrum of incident X-rays on a virtual human phantom, said incident X-rays being emitted from an X-ray source,
(b2) discretizing said X-ray spectrum,
(b3) calculating a direct X-ray intensity and a scattering process within said virtual human phantom under each energy of said discretized X-ray spectrum with density information of elements and/or electrons,
(b4) adding at least direct X-ray and scattered X-ray intensities for each X-ray energy on each detector of the X-ray flat panel, and then obtaining an X-ray projection image by performing weighted summation for all the X-ray energies according to the spectrum intensity as a function of X-ray energies.

5. The method according to claim 4, wherein the step (b1) is initially conducted without placing a bowtie filter near the X-ray source, and then said X-ray spectrum is further adjusted by the shape and material information of a bowtie filter placed near the X-ray source, thereby generating bowtie-filtered cone-angle dependent X-ray spectrums on a virtual human phantom.

6. A method for calculating density images in a human body, comprising:
(a) generating density images of electrons and/or elements for a large number of virtual human phantoms,
(b) calculating X-ray projection images of said large number of virtual human phantoms,
(c) reconstructing cone-beam CT images using said X-ray projection images,
(d) performing deep learning for a multi-layered neural network using said cone-beam CT images as input training data and said density images as output training data,
(e) obtaining density images of a new human body by inputting cone-beam CT images of said new human body to the trained multi-layered neural network.

7. The method according to claim 6, wherein the step (b) further comprising:
(b1) determining an X-ray standard spectrum model of an X-ray source, and generating a large number of X-ray spectrums by varying the parameters of said X-ray standard spectrum model,
(b2) discretizing each of said large number of X-ray spectrums,
(b3) calculating a direct X-ray intensity and a scattering process within said virtual human phantom under each energy of said discretized X-ray spectrum with density information of elements and/or electrons,
(b4) adding at least direct X-ray and scattered X-ray intensities for each X-ray energy on each detector of the X-ray flat panel, and then obtaining an X-ray projection image by performing weighted summation for all the X-ray energies according to the spectrum intensity as a function of X-ray energies.

8. The method according to claim 7, wherein the step (b1) is initially conducted without placing a bowtie filter near the X-ray source, and then said generated large number of X-ray spectrums are further adjusted by the shape and material information of a bowtie filter placed near the X-ray source, thereby generating bowtie-filtered cone-angle dependent X-ray spectrums on a virtual human phantom.

9. The method according to claim 6, wherein the step (b) further comprising:
(b1) calculating an X-ray spectrum of incident X-rays on a virtual human phantom, said incident X-rays being emitted from an X-ray source,
(b2) discretizing said X-ray spectrum,
(b3) calculating a direct X-ray intensity and a scattering process within said virtual human phantom under each energy of said discretized X-ray spectrum with density information of elements and/or electrons,
(b4) adding at least direct X-ray and scattered X-ray intensities for each X-ray energy on each detector of the X-ray flat panel, and then obtaining an X-ray projection image by performing weighted summation for all the X-ray energies according to the spectrum intensity as a function of X-ray energies.

10. The method according to claim 9, wherein the step (b1) is initially conducted without placing a bowtie filter near the X-ray source, and then said X-ray spectrum is further adjusted by the shape and material information of a bowtie filter placed near the X-ray source, thereby generating bowtie-filtered cone-angle dependent X-ray spectrums on a virtual human phantom.

11. A method for calculating density images in a human body, comprising:
(a) generating density images of electrons and/or elements for a large number of virtual human phantoms,
(b) calculating X-ray projection images of said large number of virtual human phantoms,
(c) performing deep learning for a multi-layered neural network using said X-ray projection images as input training data, and a set of said density images and corresponding organ label images as output training data,
(d) obtaining a set of said density images and corresponding organ label images of a new human body by inputting projection images of said new human body to the trained multi-layered neural network.

12. The method according to claim 11, wherein the step (b) further comprising:
(b1) determining an X-ray standard spectrum model of an X-ray source, and generating a large number of X-ray spectrums by varying the parameters of said X-ray standard spectrum model,
(b2) discretizing each of said large number of X-ray spectrums,
(b3) calculating a direct X-ray intensity and a scattering process within said virtual human phantom under each energy of said discretized X-ray spectrum with density information of elements and/or electrons,
(b4) adding at least direct X-ray and scattered X-ray intensities for each X-ray energy on each detector of the X-ray flat panel, and then obtaining an X-ray projection image by performing weighted summation for all the X-ray energies according to the spectrum intensity as a function of X-ray energies.

13. The method according to claim 12, wherein the step (b1) is initially conducted without placing a bowtie filter near the X-ray source, and then said generated large number of X-ray spectrums are further adjusted by the shape and material information of a bowtie filter placed near the X-ray source, thereby generating bowtie-filtered cone-angle dependent X-ray spectrums on a virtual human phantom.

14. The method according to claim 11, wherein the step (b) further comprising:
(b1) calculating an X-ray spectrum of incident X-rays on a virtual human phantom, said incident X-rays being emitted from an X-ray source,
(b2) discretizing said X-ray spectrum,
(b3) calculating a direct X-ray intensity and a scattering process within said virtual human phantom under each energy of said discretized X-ray spectrum with density information of elements and/or electrons,
(b4) adding at least direct X-ray and scattered X-ray intensities for each X-ray energy on each detector of the X-ray flat panel, and then obtaining an X-ray projection image by performing weighted summation for all the X-ray energies according to the spectrum intensity as a function of X-ray energies.

15. The method according to claim 14, wherein the step (b 1) is initially conducted without placing a bowtie filter near the X-ray source, and then said X-ray spectrum is further adjusted by the shape and material information of a bowtie filter placed near the X-ray source, thereby generating bowtie-filtered cone-angle dependent X-ray spectrums on a virtual human phantom.

16. A method for calculating density images in a human body, comprising:
(a) generating density images of electrons and/or elements for a large number of virtual human phantoms,
(b) calculating X-ray projection images of said large number of virtual human phantoms,
(c) reconstructing X-ray cone-beam CT images using said X-ray projection images,
(d) performing deep learning for a multi-layered neural network using said X-ray cone-beam CT images as input training data, and a set of said density images and corresponding organ label images as output training data,
(e) obtaining density images and organ label images of a new human body by inputting X-ray cone-beam CT images of said new human body to the trained multi-layered neural network.

17. The method according to claim 16, wherein the step (b) further comprising:
(b1) determining an X-ray standard spectrum model of an X-ray source, and generating a large number of X-ray spectrums by varying the parameters of said X-ray standard spectrum model,
(b2) discretizing each of said large number of X-ray spectrums,
(b3) calculating a direct X-ray intensity and a scattering process within said virtual human phantom under each energy of said discretized X-ray spectrum with density information of elements and/or electrons,
(b4) adding at least direct X-ray and scattered X-ray intensities for each X-ray energy on each detector of the X-ray flat panel, and then obtaining an X-ray projection image by performing weighted summation for all the X-ray energies according to the spectrum intensity as a function of X-ray energies.

18. The method according to claim 17, wherein the step (b1) is initially conducted without placing a bowtie filter near the X-ray source, and then said generated large number of X-ray spectrums are further adjusted by the shape and material information of a bowtie filter placed near the X-ray source, thereby generating bowtie-filtered cone-angle dependent X-ray spectrums on a virtual human phantom.

19. The method according to claim 16, wherein the step (b) further comprising:
(b1) calculating an X-ray spectrum of incident X-rays on a virtual human phantom, said incident X-rays being emitted from an X-ray source,
(b2) discretizing said X-ray spectrum,
(b3) calculating a direct X-ray intensity and a scattering process within said virtual human phantom under each energy of said discretized X-ray spectrum with density information of elements and/or electrons,
(b4) adding at least direct X-ray and scattered X-ray intensities for each X-ray energy on each detector of the X-ray flat panel, and then obtaining an X-ray projection image by performing weighted summation for all the X-ray energies according to the spectrum intensity as a function of X-ray energies.

20. The method according to claim 19, wherein the step (b1) is initially conducted without placing a bowtie filter near the X-ray source, and then said X-ray spectrum is further adjusted by the shape and material information of a bowtie filter placed near the X-ray source, thereby generating bowtie-filtered cone-angle dependent X-ray spectrums on a virtual human phantom.
